(19) United States
(12) Patent Application Publication
(10) Pub. No.: US 2008/0299189 A1
(43) Pub. Date: Dec. 4, 2008

(54) CONTROLLED RELEASE DOPAMINE AGONIST COMPOSITIONS

(75) Inventors: Jonathan David Bortz, Saint Louis, MO (US); Michael Grimshaw, Saint Louis, MO (US); David F. Erkoboni, Pennington, NJ (US)

Correspondence Address: KV PHARMACEUTICAL COMPANY, 4080B WEDGEWAY COURT, EARTH CITY, MO, 63045, US

(73) Assignee: DRUGTECH CORPORATION, Wilmington, DE (US)

(21) Appl. No.: 12/126,575

(22) Filed: May 23, 2008

Related U.S. Application Data
(60) Provisional application No. 60/933,271, filed on Jun. 4, 2007.

(51) Int. Cl. A61K 9/52; A61K 9/24; A61K 31/428; A61P 25/16

(52) U.S. Cl. 424/457; 424/472; 514/367

(57) ABSTRACT

The present invention relates to a multiphase release oral pharmaceutical formulation having a dopamine agonist as an active ingredient. The multiphase composition comprises at least two different release components. The invention relates to controlled release pharmaceutical compositions of pramipexole or a pharmaceutically acceptable salt thereof for once-daily administration.

This application claims the benefit of U.S. provisional patent application Ser. No. 60/933,271, filed on Jun. 4, 2007, the entire disclosure of which is incorporated by reference herein.

### FIELD OF THE INVENTION

The present invention relates to controlled release pharmaceutical compositions having a dopamine receptor agonist as an active ingredient. More specifically, the invention relates to controlled release pharmaceutical compositions of pramipexole or a pharmaceutically acceptable salt thereof. Still more specifically, the invention relates to controlled release pharmaceutical compositions of pramipexole or a pharmaceutically acceptable salt thereof for once-daily administration.

### BACKGROUND OF THE INVENTION

An estimated 1 million Parkinson's Disease patients exist in the United States, of which approximately 200,000 patients are taking medications that are not achieving the desired therapeutic benefits. Parkinson's Disease is the 2nd most common neurodegenerative disease, costing an estimated $27 billion annually. About 60,000 new cases of Parkinson's Disease are diagnosed annually, and it has been demonstrated that the starting therapeutic regimen may have an impact on the long term outcomes of these patients. Parkinson's Disease is rare in individuals less than 40 years of age, but is present in more than 1% of people over age 60 and 2% of people over age 85. As the population ages, the incidence of Parkinson's Disease is expected to increase.

Parkinson's Disease is associated with a loss of dopamine producing cells in a part of the brain called the basal ganglia (more specifically, the substantia nigra), as well as a degenerative component that is not yet clearly elucidated. The main clinical effects of the lack of dopamine as a neurotransmitter are a resting tremor, stiffness and cogwheel rigidity, postural instability and bradykinesia.

The cornerstone of therapy has been the administration of levodopa (the precursor of dopamine), which was initially developed in the 1960s. Unfortunately, because levodopa is rapidly decarboxylated to dopamine in the periphery, very little can get through the blood brain barrier. As a consequence, large doses must be given, which give rise to significant side effects because, even though the blood brain barrier was impervious to dopamine, the vomiting center is not, and dopamine in the periphery stimulates the vomiting center to produce severe side effects. In the 1980s, carbidopa was added to levodopa because it inhibits the peripheral decarboxylation, making more levodopa available to the brain and reducing the dose of levodopa that was initially used by 75%. However, the short elimination half-life of 1.5 hours meant that dosing had to take place every 3 to 4 hours to achieve a clinical effect. Despite this frequent dosing regimen, after 3 to 5 years, most patients develop complications including shortening of efficacy (wearing off effect), and dyskinesias (involuntary ‘choreaform’ movements). Many patients began cycling between these two complications, several times per day.

Another class of pharmacologic agents has been developed that do not rely on any enzymatic activity in the brain to increase the amount of dopamine available as a neurotransmitter. These agents stimulate the dopamine receptor directly and are called dopamine agonists. These agents can be used with levodopa/carbidopa or in place of it. Although dopamine agonists have less motor complications, they may have other side effects such as somnolence and hallucinations. Furthermore, because of the short half-life of several of these medications (pramipexole 8.5 hours with a Cₘₐₓ of 1-3 hours), the intermittent dosing of the agonists has led to a pulsatile blood profile. It has been shown that continuous delivery (or steadier blood levels) of dopamine agonists have better clinical responses with much less dyskinesia. Therefore, many investigators have recommended that using a compound like pramipexole should be considered for initiation of therapy in mild to severe Parkinson's disease, alone or in combination with levodopa. In addition, some researches have suggested that the progression of Parkinson's may be slowed down with early introduction of dopamine agonists.

Pramipexole is chemically designated as (S)-2-amino-4,5,6,7-tetrahydro-6-(propylamino)benzothiazole and has the molecular formula C₁₀H₁₇N₃S. The chemical structure of pramipexole is:

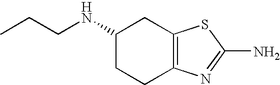

Pramipexole as its dihydrochloride salt is commercially available as Mirapex® tablets (Boehringer Ingelheim). These are immediate-release tablets in 0.125 mg, 0.25 mg, 0.5 mg, 1.0 mg and 1.5 mg strengths, designed for oral administration three times daily to provide a total dose of 0.375 to 4.5 mg per day. Although the three times daily dosing regimen for Mirapex tablets is well tolerated, patient compliance may be greatly improved if a once-daily regimen were available.

Despite the benefits of dopamine agonist therapy, this class of pharmaceutical agents have three significant drawbacks which may be addressed by this invention. The adverse events that may occur during dopamine agonist therapy include dizziness, nausea, hypotension, hallucinations and somnolence. Pramipexole's most frequent side effect is hallucination with relative risk of 5.2 when compared to placebo. Pramipexole's relative risk of somnolence was 2.01 compared to placebo, and pramipexole's relative risk of hypotension was 1.65 compared to placebo.

Most patients require 1.5 mg or less per day of pramipexole to achieve clinical benefit, and most side effects are seen with doses in excess of this. A daily regimen of 1.5 mg or less results in fewer side effects. Extending the delivery of pramipexole and thereby creating a more continuous blood level profile may improve clinical efficacy and reduce dyskinesias even further and possibly other side effects as well. Also, the rate of early drop-outs may potentially be mitigated by a carefully structured initiation regimen designed to start the patient at a lower dose and titrate to an effective clinical dose. This may reduce the number of patients who begin this therapy and discontinue early because of poorly tolerated side effects early on.

Dopamine agonists are also effective in the treatment of restless legs syndrome (RLS). RLS is a disease that is characterized by a compelling urge to move the legs that is usually accompanied, or caused, by uncomfortable and unpleasant sensations in the legs. Because the sensations that accompany RLS are unusual, patients may have a hard time describing them. Patients often use words like uncomfortable, creeping, itching, burning, pulling, or creepy-crawly to describe feelings inside the leg. It is not uncommon for the sensations to spread to the arms or other body parts, in addition to the legs. The symptoms begin or worsen during periods of rest or inactivity, such as lying or sitting. The symptoms are partially or completely relieved by movement, as long as the movement continues. The urge to move may be irresistible, and often the only way to stop or partially relieve the sensations is to move around. However, the relief is not always complete and may end when the activity ends. While the cause of primary restless legs syndrome is unknown, nearly half of the time it can be traced to a family history. Although there is currently no cure for primary restless legs syndrome, symptoms may often be treated by administration of dopamine agonists.

The invention harnesses the beneficial effects of dopamine agonist therapy and addresses the challenges most commonly associated with this class of medication in unique dosage forms, which may be delivered in a controlled delivery system. The dosage system may be initiated in a titration regimen designed to gain quicker clinical benefits with a reduction in unwanted side effects.

### SUMMARY OF THE INVENTION

The present invention provides pharmaceutical compositions for the controlled release of a dopamine agonist. In certain embodiments, the dopamine agonist is pramipexole, which may be present as its pharmaceutically acceptable salt.

The invention provides a multiphase-release oral pharmaceutical formulation comprising: a first component comprising a dopamine agonist or a pharmaceutically acceptable salt thereof, and a second component comprising the dopamine agonist or a pharmaceutically acceptable salt thereof, wherein the first component provides a first release profile of the dopamine agonist following oral administration and the second component provides a second release profile of the dopamine agonist following oral administration. In certain embodiments, the multiphase-release oral pharmaceutical formulation comprises a first component that provides an immediate release of the dopamine agonist following oral administration, and a second component that provides a modified release of the dopamine agonist following oral administration.

In another embodiment of the invention, the multiphase-release oral pharmaceutical formulation comprises a first component that provides a pH-independent modified release of the dopamine agonist following oral administration, and a second component that provides a pH-dependent modified release of the dopamine agonist following oral administration.

In certain embodiments of the multiphase release oral pharmaceutical composition, the first component is one layer of a multi-layer tablet and the second component is another layer of the multi-layer tablet.

In one embodiment of the multiphase release oral pharmaceutical composition, the first component may be a first group of immediate-release beads and the second component may be a second group of modified release beads. In another embodiment of the multiphase release oral pharmaceutical composition, the first component may be a first group of controlled-release beads and the second component may be a second group of controlled-release beads. Alternatively, the first component and the second component are combined on a single group of controlled release beads. The controlled release beads may be compressed into a tablet or may be contained in a capsule.

In preferred embodiments, the invention provides a multiphase release oral pharmaceutical composition that upon ingestion provides a rapid initial increase in the blood plasma concentration of the dopamine agonist and a relatively steady blood plasma concentration thereafter.

The multiphase release oral pharmaceutical composition preferably provides a controlled release of the dopamine agonist wherein at least about 15%, and more preferably at least about 20%, of the total dopamine agonist or pharmaceutically acceptable salt thereof is released from the formulation within the first 1.5 hours, and most preferably within the first hour, as measured in an in vitro dissolution test. Preferably, the formulation further provides a controlled release of dopamine agonist wherein about 40% of the total dopamine agonist is released from the composition within the first three hours, and more preferably within the first two hours, as measured in an in vitro dissolution test.

In another embodiment, the multiphase release oral pharmaceutical composition comprises a dopamine agonist or a pharmaceutically acceptable salt thereof, and a pharmaceutically acceptable excipient, said composition providing an in vitro release profile wherein at least about 15%, and more preferably at least about 20%, of the dopamine agonist is released within the first 1.5 hours, and most preferably within the first hour, as measured in an in vitro dissolution test, and wherein said composition provides an in vivo absorption profile wherein the time to reach 30% absorption, and more preferably 40% absorption, is about 3 hours or less on average after a single dose oral administration to a human adult.

The invention also provides a method of treating a condition in a patient by administering to the patient a multiphase release oral pharmaceutical composition described herein. Preferably, the condition is selected from Parkinson's Disease and restless leg syndrome, sleep disorders, sleep related eating disorder, cocaine and other chemical dependency, clinical depression, erectile dysfunction, ischemic, neurodegenerative diseases and fibromyalgia.

### DETAILED DESCRIPTION OF THE INVENTION

The invention provides a multiphase release oral pharmaceutical formulation having a dopamine agonist as an active ingredient. The multiphase composition comprises at least two different release components. Upon oral administration, the multiphase release oral pharmaceutical formulations of the invention provide a rapid increase in the blood plasma concentration of the dopamine agonist followed by a relatively steady blood plasma concentration for a sustained period, preferably at least about 18 to 24 hours. Thus, the multiphase release oral pharmaceutical formulations of the invention may allow rapid onset of the pharmaceutical effect of the active agent while avoiding peaks and troughs in blood plasma concentration. Peaks in the blood plasma level may be associated with increased incidence of adverse effects. Conversely, troughs in the blood plasma concentration may be associated with less efficient, or insufficient, control of the patient's symptoms.

In one embodiment, a first component comprises a dopamine agonist or a pharmaceutically acceptable salt thereof, and provides an immediate release of the dopamine agonist following oral administration to a patient. A second component comprises a dopamine agonist or a pharmaceutically acceptable salt thereof and provides a modified release of the dopamine agonist following oral administration to a patient.

In another embodiment, a first component comprises a dopamine agonist or a pharmaceutically acceptable salt thereof, and provides a pH-independent modified release of the dopamine agonist following oral administration to a patient. A second component comprises a dopamine agonist or a pharmaceutically acceptable salt thereof and provides a pH-dependent modified release of the dopamine agonist following oral administration to a patient.

The term “multiphase release” as used herein refers to a pharmaceutical composition that comprises two or more drug delivery components, each of which have a dopamine agonist, for example, pramipexole or a pharmaceutically acceptable salt thereof, as the active ingredient and each of which release the dopamine agonist with a different release profile. The release profile of each component may be selected from immediate release or modified release. The modified release may be pH-independent or may be pH-dependent.

The dopamine agonist may be selected from, for example, apomorphine, bromocriptine, cabergoline, dostinex, dihydroergocryptine, fenoldam, lisuride, lysergic acid, sumanirole, pergolide, piribedil, pramipexole, quinpirole, ropinirole, rotigotine, SKF 38393, SKF 82958, or pharmaceutically acceptable salts or enatiomers thereof. In preferred embodiments, the dopamine agonist is pramipexole or a pharmaceutically acceptable salt thereof. In particularly preferred embodiments, the dopamine agonist is pramipexole dihydrochloride.

In a preferred embodiment of the invention, one component of the multiphase release provides an immediate release of pramipexole or pharmaceutically acceptable salt thereof subsequent to administration and the other component provides a pH-dependent release. In one embodiment, the two components may be immediate/controlled release beads, wherein the first component is a portion of the beads that provide an immediate release of pramipexole and the second component is another portion of the beads that provides a modified release of pramipexole. In another embodiment, instead of having two groups of different beads, the first and second components may be combined into a single type of bead, for example, a bead with an inner layer (or core) and an outer layer. In either case, the beads may be compressed into a tablet or filled into a capsule. In another embodiment, the two components may be the separate portions of a multi-layer tablet, with one layer providing the immediate release of pramipexole and the other layer providing the modified release of pramipexole.

In a another embodiment of the invention, one component of the multiphase release provides an pH-independent modified release of pramipexole or pharmaceutically acceptable salt thereof subsequent to administration and the other component provides a pH-dependent modified release. In one embodiment, the two components may be controlled release beads, wherein the first component is a portion of the beads that provide a pH-independent modified release of pramipexole and the second component is another portion of the beads that provides a pH-dependent modified release of pramipexole. In another embodiment, instead of having two groups of different beads, the first and second components may be combined into a single type of bead, for example, a bead with an inner layer (or core) and an outer layer. In either case, the beads may be compressed into a tablet or filled into a capsule. In another embodiment, the two components may be the separate portions of a multi-layer tablet, with one layer providing the pH-independent modified release of pramipexole and the other layer providing the pH-dependent modified release of pramipexole.

In another embodiment of the invention, the multiphase release composition may have three drug delivery components. In this embodiment, the composition may comprise a first phase which is immediate release, a second phase which is pH-independent modified release and a third phase which has a pH-dependent modified release. In one embodiment, the three components may be immediate/controlled release beads, wherein the first component is a portion of the beads that provide an immediate release of pramipexole, the second component is another portion of beads that provide a pH-independent modified release of pramipexole, and the third component is another portion of the beads that provides a pH-dependent modified release of pramipexole.

The term “modified release” as used herein refers to a release from a pharmaceutical formulation that occurs over an extended period of time, occurs after a delay, or both, as compared to a conventional, immediate release formulation of the same drug. An “immediate release” dosage has a release of the active substance that is not deliberately modified by a special formulation design and/or manufacturing method and therefore occurs as quickly as is consistent with the properties of the active substance. By contrast, the modified release formulation is adapted in order to retard the release of all of the active substance or at least a substantial portion of the active substance.

The term “pH-dependent” as used herein refers to a release to the active substance in a manner that is substantially dependent upon the pH of the surrounding medium within the pH ranges normally found in the human GI tract. A pH-dependent release component may be designed to provide substantially no release or a slow release of the active agent at low pH (i.e., the pH of the stomach) and a higher rate of release at a higher pH (i.e., the pH of the intestines). Such components may be referred to as “delayed release.” By contrast, a pH-independent formulation provides release of the active substance that is substantially independent of the pH of the surrounding medium within pH ranges normally found in the human GI tract.

An immediate release component may comprise from about 1% to about 20% of the total dopamine agonist in the dosage unit. The immediate release component may further comprise binders, disintegrants, lubricants, etc.

A modified release component may comprise from about 20% to about 99% of the total dopamine agonist in the dosage unit. The modified release component comprises one or more means of slowing the release of the dopamine agonist from the component. In one embodiment, the modified release component may comprise an erodable matrix in which the dopamine agonist is dispersed. The modified release component may be obtained by preparing a hydrogel-type matrix using a water-soluble polymer as a release sustaining material. Gel-forming polymers include, for example, hydrophilic polymers such as hydroxypropyl methylcellulose, hydroxypropyl cellulose, methyl cellulose, hydroxyethyl cellulose, polyvinyl pyrrolidone, and polyethylene oxides, carbomer, carboxymethylcellulose, methacrylate polymers and copolymers, psyllium and natural gums such as guar gum, and arabic gum, gelatin, locust bean, acacia, carrageenan, sodium alginate, algininc acid, xanthan. The hydrophilic gel-forming polymers can be used alone or in combination with other hydrophilic polymers such as propylene glycol, polyethylene glycol, microcrystalline cellulose, croscarmellose, starch, chitosan, methacrylate polymers, dextrin, maltodextrin, dextran, glucomannans; galactomannans, pectin, block copolymers of ethylene oxide and propylene oxide, polyvinyl alcohol or in combination with hydrophobic polymers such as ethylcellulose, chitin, cellulose acetate, cellulose acetate butyrate, cellulose acetate phthalate, cellulose acetate propionate, glyceryl behenate, glycerol paimetostearate, glycerol monostearate, waxes including carnuba, bees, microcrystalline, methacrylate polymers and copolymers, polyvinyl acetate, and sulfonated divinylbenzene/styrene copolymer.

Alternatively, the modified release component may be coated with a material that functions to slow the release of the dopamine agonist from the component. Such coatings include but are not limited to, for example, ethylcellulose and polyvinylacetate, cellulose acetate, cellulose acetate phthalate, cellulose acetate butarate, shellac, methacrylate polymers and copolymers, hydroxypropylmethcellulose phthalate and waxes. The insoluble coatings may be used in conjunction with water soluble polymers and compounds to increase the permeability of the insoluble film. Such polymers and compounds include hydroxypropyl methylcellulose, hydroxypropyl cellulose, methyl cellulose, hydroxyethyl cellulose, polyvinyl pyrrolidone, polyethylene oxides, carbomer, carboxymethylcellulose, methacrylate polymers and copolymers, guar gum, and arabic gum, gelatin, locust bean, acacia, carrageenan, sodium alginate, algininc acid, xanthan, sucrose, lactose, propylene glycol, polyethylene glycol, dextrin, maltodextrin, dextran, glucomannans; galactomannans, pectin, block copolymers of ethylene oxide and propylene oxide, polyvinyl alcohol polysorbates, sodium lauryl sulfate. The coating layers may also contain pharmaceutically acceptable plasticizers, dispersants, colorants, anti-tack and anti-foaming agents.

A pH-dependent component may comprise from about 20% to about 99% of the total dopamine agonist in the dosage unit. The pH-dependent component also comprises at least one means of controlling the release of the dopamine agonist based on the pH of the surrounding media. In preferred embodiments, the pH-dependent component will release little or no dopamine agonist at low pH (i.e., in the stomach), but will begin to release the dopamine agonist once the dosage unit reaches the higher pH environment of the intestines.

The pH-dependent component may be coated with an enteric coating. Such enteric coatings are enteric polymeric materials containing weakly acidic functional groups, which are capable of ionization at elevated pH. In the low pH of the stomach, the enteric polymers are un-ionized, and therefore are insoluble. As the pH increases in the intestinal tract, these functional groups ionize, and the polymer becomes soluble in the intestinal fluids. The enteric coating may comprise cellulose acetate phthalate, hydroxypropyl methylcellulose phthalate, polyvinyl acetate phthalate, hydroxypropyl methylcellulose acetate succinate, methacrylic acid ester copolymers, or other enteric polymers known in the art. The pH at which the enteric coat will dissolve can be controlled by the selection of the enteric polymer, by the combination of polymers selected and/or by the ratio of weakly acidic groups. For example, dissolution characteristics of the enteric polymer can be adjusted by the ratio of free carboxyl groups to ester groups.

Enteric coating layers may also contain pharmaceutically acceptable plasticizers such as triethyl citrate, dibutyl phthalate, and polyethylene glycols, or other plasticizers. Additives such as dispersants, colorants, anti-tack and anti-foaming agents may also be included.

Alternatively, the pH-dependent component may comprise the dopamine agonist dispersed in a polymer matrix, wherein the polymer matrix comprises one or more polymers that allow for release of the dopamine agonist in a pH-dependent manner. Such polymers include, for example, cellulose acetate phthalate, hydroxypropyl methylcellulose phthalate, polyvinyl acetate phthalate, sodium alginate hydroxypropylmethylcellulose acetate succinate, methacrylic acid copolymers, and polymethyl vinyl ether /maleic acid copolymer.

In certain embodiments, the controlled release pharmaceutical compositions may be comprised of controlled-release granules or beads, which may be compressed into a tablet or filled in a capsule. The composition may further comprise a portion of immediate release beads.

Immediate release beads may be prepared by dissolving the dopamine agonist or its pharmaceutically acceptable salt in a suitable solvent. Suitable solvents include, but are not limited to, water, methanol and ethanol. Preferably, the resulting solution has a concentration ranging from about 0.01% to about 0.15% (w/w). The solution is mixed with one or more granulation binders, and optionally, other excipients as described herein may also be added. Granulations binders include, but are not limited to PVP, hydroxypropyl methylcellulose, hydroxyethyl cellulose, hydroxypropyl cellulose and starch. Other excipients as described herein may also be added. The mixture is granulated in a granulator, for example using a high shear granulator (e.g. Fluid Air High shear granulator with various bowl sizes) or a fluid bed granulator (e.g. Fluid Air PX50). The resulting granules are dried. The granules may be oven dried at an elevated temperature for a period of time (i.e., for 24 hrs), or dried using a fluid-bed drier.

In one embodiment, modified release beads are formed by coating immediate release beads with a coating that slows the release of the dopamine agonist from the bead. Examples of such coatings include ethylcellulose and polyvinylacetate.

Alternatively, a modified release bead may be prepared by mixing a solution of the dopamine agonist, or a pharmaceutically acceptable salt, with release-controlling polymers and optionally a granulation binder and other excipients. Suitable solvents include, but are not limited to, water, methanol, and ethanol. Preferably, the resulting solution has a concentration ranging from about 0.01% to about 15% (w/w). Granulations binders include, but are not limited to PVP, hydroxypropyl methylcellulose, hydroxyethyl cellulose, hydroxypropyl cellulose and starch. The release-controlling polymer may be selected from, for example, hydroxypropyl methylcellulose, hydroxypropyl cellulose, polyvinyl pyrrolidone, and polyethylene oxides, and natural gums such as guar gum, and arabic gum, or a combination of polymers. The solution of the dopamine agonist may be mixed with the release-controlling polymer and other excipients in a granulator, such as a high shear granulator (e.g. Fluid Air High shear granulator with various bowl sizes) or a fluid bed granulator (e.g. Fluid Air PX50). The granules may be oven dried at an elevated temperature for a period of time (i.e., for 24 hrs), or dried using a fluid-bed drier.

In one embodiment, delayed release beads are formed by coating immediate release beads with a coating that delays the release of the dopamine agonist from the bead. Examples of such coatings include enteric coatings such as cellulose acetate phthalate, hydroxypropyl methylcellulose phthalate, polyvinyl acetate phthalate, hydroxypropyl methylcellulose acetate succinate, methacrylic acid ester copolymers, or other enteric polymers known in the art.

Alternatively, a delayed release bead may be prepared by mixing a solution of the dopamine agonist, or a pharmaceutically acceptable salt, with enteric polymers and optionally a granulation binder and other excipients. Suitable solvents include water, methanol, ethanol, etc. Preferably, the resulting solution has a concentration ranging from about 0.1% to about 15% (w/w). Granulations binders include, but are not limited to PVP, hydroxypropyl methylcellulose, hydroxyethyl cellulose, hydroxypropyl cellulose and starch. The enteric polymer may be selected from, for example, cellulose acetate phthalate, hydroxypropyl methylcellulose phthalate, polyvinyl acetate phthalate, and sodium alginate, or a combination of polymers. The solution of the dopamine agonist may be mixed with the enteric polymer(s) and other excipients in a granulator, such as a high shear granulator (e.g. Fluid Air High shear granulator with various bowl sizes) or a fluid bed granulator (e.g. Fluid Air PX50). The granules may be oven dried at an elevated temperature for a period of time (i.e., for 24 hrs), or dried using a fluid-bed drier.

In certain embodiments, a multilayer tablet may be prepared by mixing multiple granule types (immediate release granules, slow release granules, and/or delayed release granules). For example, a bilayer tablet may be prepared using a combination of slow release granules and delayed release granules. The granules are blended, preferably with the addition of a suitable lubricant, and compressed. Suitable lubricants include, but are not limited to, magnesium stearate, sodium stearyl fumarate, stearic acid, and sodium starch glycolate. The tablets may be compressed to a tablet hardness of between about 4 to about 18 kp (about 5.6 to about 25.2 SC). Preferably, the press is capable of making bilayer tablets, such as Picola and Korsch bi-layer presses.

The term “patient” as used herein refers to any animal in need of treatment with the composition of the invention. The term patient preferably refers to mammals and most preferably to humans.

The compositions of the present invention may be used in the treatment of, for example, Parkinson's Disease, restless leg syndrome, cluster headache, and bipolar disorder. The pharmaceutical composition of this invention may be orally administered in any acceptable dosage form, most preferably capsules and tablets.

The invention also provides a method of treating a condition in a patient, preferably a human, by administering to the patient an effective amount of the composition of the present invention. The composition of the invention may be administered alone or may be administered in combination one or more of other approved therapeutics, such as other antiparkinsonian medications including, for example, levodopa, carbadopa, MAO-B inhibitors (such as selegiline, deprenyl, and rasagiline), COMT inhibitors (such as entacapone, tolcapone and nitecapone), amantadine and rivastigmine. Alternatively, these additional agents may be separately administered to a patient.

The dosage of pramipexole may vary, depending on the reason for use and the individual patient. The dosage may be adjusted based on the patient's body weight and based on the patient's tolerance of the dopamine agonist. For pramipexole, each dosage unit may comprise 0.125 mg, 0.25 mg, 0.37mg, 0.5 mg, 1 .0 mg, 1.5 mg, 3 mg, and 4.5 mg strengths.

Compositions in accordance with the invention may also contain other ingredients. For example, the composition may include, in addition to the foregoing, one or more other ingredients that are included as binders, lubricants, disintigrants, diluents, anti-oxidants, flavoring agents, coloring agents, and the like. Other suitable excipients for the compositions can be found in standard pharmaceutical texts, e.g. in “Remington's Pharmaceutical Sciences,” The Science and Practice of Pharmacy, 19th Ed. Mack Publishing Company, Easton, Pa., (1995).

Upon oral administration, the multiphase release oral pharmaceutical formulations of the invention provide a rapid increase in the blood plasma concentration of the dopamine agonist. To this end, the multiphase release oral pharmaceutical composition preferably provides a controlled release of the dopamine agonist wherein at least about 15%, and more preferably at least about 20%, of the total dopamine agonist or pharmaceutically acceptable salt thereof is released from the formulation within the first 1.5 hours, and most preferably within the first hour, as measured in an in vitro dissolution test. Preferably, the formulation further provides a controlled release of dopamine agonist wherein about 40% of the total dopamine agonist is released from the composition within the first three hours, and more preferably within the first two hours, as measured in an in vitro dissolution test.

In another embodiment, the multiphase release oral pharmaceutical composition comprises a dopamine agonist or a pharmaceutically acceptable salt thereof, and a pharmaceutically acceptable excipient, said composition providing an in vitro release profile wherein at least about 15%, and more preferably at least about 20%, of the dopamine agonist is released within the first 1.5 hours, and most preferably within the first hour, as measured in an in vitro dissolution test, and wherein said composition provides an in vivo absorption profile wherein the time to reach 30% absorption, and more preferably 40% absorption, is about 3 hours or less on average after a single dose oral administration to a human adult.

The “dissolution test” as used herein refers to the in vitro dissolution test using Apparatus I (Basket Apparatus) (United States Pharmacopoeia, 29 (2006) pp. 2675-2682) and a dissolution media of 900 ml at 37° C. and 100 rpm. In the first two hours of the dissolution, a dissolution media of pH 1.5 is used, and switch to a media of pH 6.8 for the subsequent 3 to 24 hours dissolution testing

The compositions of the present invention may be prepared according to conventional methods. For example, matrix-type components or tablets may be manufactured by direct compression of a dry mixture of the dopamine agonist, the matrix polymer, and other optional excipients.

A multi-layer tablet may be manufactured by (i) blending a quantity of the dopamine agonist with various excipients to form the rapid releasing component, (ii) blending another quantity of the dopamine agonist with a hydrophilic polymer(s) and/or a water-insoluble polymer(s) and various excipients to form a sustained release component, and (iii) blending another quantity of the dopamine agonist with an enteric polymer(s) and various excipients to form the pH-dependent component; and compressing a quantity of the rapid release component, a quantity of the slow release component, and a quantity of the pH-dependent component to form a multi-layer tablet. The tablet may then be optionally coated with a protective coating.

The coating layers (sustained, enteric, protective, etc.) may be applied onto the core using standard coating techniques. The coating may be applied by dissolving or suspending the coating material(s) in a suitable medium, such as water, methanol, ethanol, acetone, ethyl acetate, methylene chloride, etc., or mixtures thereof, and the resultant solution or suspension may be sprayed onto the core to coat them, followed by drying, for example in an air flow. In certain embodiments, the coating material may be dissolved or suspended in a solvent and coated onto the core using a fluidized bed system.

### EXAMPLES

The formulations listed below are intended to represent possible pramipexole HCI compositions of the present invention. It is understood that the materials and amounts do not necessarily limit the scope of the invention.

### Example 1

### 0.375 mg Pramipexole HCl Bilayer Tablet

ER Layer

| Composition Percentage | (%) |
|---|---|
| Pramipexole | 0.18 |
| Starch | 5-25% |
| Sodium Alginate | 20-60% |
| PVP | 1-5% |
| HPMC | 20-60% |
| Magnesium Stearate | 0.5 |

IR Layer

| Composition Percentage | (%) |
|---|---|
| Pramipexole | 0.0875 (0.001-0.1%) |
| Starch | 50-90% |
| Microcrystalline Cellulose | 10-40% |
| PVP | 1.25 (1-5%) |
| Magnesium Stearate | 0.5 |

The ER layer granulation may be prepared by dissolving 0.72 g of pramipexole hydrochloride in 100 g of water to make 0.72% (w/w) pramipexole solution. PVP is added to the solution. Starch, sodium alginate and HMPC are granulated with the solution in a granulator. A high shear granulator (e.g. Fluid Air High shear granulator with various bowl sizes) or a fluid bed granulator (e.g. Fluid Air PX50) may be used. The granules may be oven dried at 45° C. for 24 hrs, or dried using a fluid-bed drier.

The IR layer granulation may be prepared by dissolving 0.025 g of pramipexole hydrochloride in 100 g of water to make 0.72% (w/w) pramipexole solution. PVP is added to the solution. Starch and microcrystalline cellulose are granulated with the solution in a granulator. A high shear granulator (e.g. Fluid Air High shear granulator with various bowl sizes) or a fluid bed granulator (e.g. Fluid Air PX50) may be used. The granules may be oven dried at 45° C. for 24 hrs, or dried using a fluid-bed drier.

A mixture of the ER granules and IR granules are blended with about 0.25-3% of magnesium stearate. The granules are compressed into a bi-layer tablet on a bi-layer press, such as Picola and Korsch bi-layer presses. The tablet hardness may be between 4 to 18 kp (5.6-25.2 SC).

### Example 2

A bilayer tablet may be prepared according to the general procedure set forth in Example 1, but having the composition of the ER layer as provided below:

ER Layer

| Composition Percentage | (%) |
|---|---|
| Pramipexole | 0.18 |
| Starch | 19.32 |
| Ethylcellulose | 20.0 |
| HPMC | 60.0 |
| Magnesium Stearate | 0.50 |

### Example 3

A bilayer tablet may be prepared according to the general procedure set forth in Example 1, but having the composition of the ER layer as provided below:

ER Layer

| Composition Percentage | (%) |
|---|---|
| Pramipexole | 0.18 |
| Starch | 19.32 |
| Eudragit | 20.0 |
| HPMC | 60.0 |
| Magnesium Stearate | 0.50 |

### Example 4

A bilayer tablet may be prepared according to the general procedure set forth in Example 1, but having the composition of the ER layer as provided below:

ER Layer

| Composition Percentage | (%) |
|---|---|
| Pramipexole | 0.18 |
| Dibasic Calcium Phosphate | 38.07 |
| PVP | 1.25 |
| HPMC | 60.0 |
| Magnesium Stearate | 0.50 |

### Example 5

A bilayer tablet may be prepared according to the general procedure set forth in Example 1, but having the composition of the ER layer as provided below:

ER Layer

| Composition Percentage | (%) |
|---|---|
| Pramipexole | 0.18 |
| Starch | 38.07 |
| PVP | 1.25 |
| HPC | 60.0 |
| Magnesium Stearate | 0.50 |

### Example 6

A bilayer tablet may be prepared according to the general procedure set forth in Example 1, but having the composition of the ER layer as provided below:

ER Layer

| Composition Percentage | (%) |
|---|---|
| Pramipexole | 0.18 |
| Starch | 19.32 |
| Ethylcellulose | 20.0 |
| HMPC | 60.0 |
| Magnesium Stearate | 0.50 |

### Example 7

A bilayer tablet may be prepared according to the general procedure set forth in Example 1, but having the composition of the ER layer as provided below:

ER Layer

| Composition Percentage | (%) |
|---|---|
| Pramipexole | 0.18 |
| Starch | 18.07 |
| Ethylcellulose | 20.0 |
| HMPC | 60.0 |
| PVP | 1.25 |
| Magnesium Stearate | 0.50 |

### Example 8

A bilayer tablet may be prepared according to the general procedure set forth in Example 1, but having the composition of the ER layer as provided below:

ER Layer

| Composition Percentage | (%) |
|---|---|
| Pramipexole | 0.18 |
| Dibasic Calcium Phosphate | 58.07 |
| Sodium Alginate | 40.0 |
| PVP | 1.25 |
| Magnesium Stearate | 0.50 |

### Example 9

A bilayer tablet may be prepared according to the general procedure set forth in Example 1, but having the composition of the ER layer as provided below:

ER Layer

| Composition Percentage | (%) |
|---|---|
| Pramipexole | 0.18 |
| Starch | 7.32 |
| Sodium Alginate | 40.0 |
| HPC | 50.0 |
| PVP | 2.00 |
| Magnesium Stearate | 0.50 |

1. A multiphase release oral pharmaceutical formulation comprising: a first component comprising a dopamine agonist or a pharmaceutically acceptable salt thereof, and a second component comprising a dopamine agonist or a pharmaceutically acceptable salt thereof, wherein the first component provides an immediate release of the dopamine agonist or the pharmaceutically acceptable salt thereof following oral administration to a patient, and wherein the second component provides a modified release of the dopamine agonist or the pharmaceutically acceptable salt thereof following oral administration to a patient.

2. The formulation of claim 1, wherein the first components is one layer of a multi-layer tablet and the second component is another layer of the multi-layer tablet.

3. The formulation of claim 1, wherein the first components comprises immediate release beads and the second component comprises controlled-release beads.

4. The formulation of claim 1, wherein the first component and the second component are combined on a single group of controlled release beads.

5. The formulation of claim 3 or 4, wherein the beads are compressed into a tablet.

6. The formulation of claim 3 or 4, wherein the beads are contained in a capsule.

7. The formulation of claim 1, wherein the dopamine agonist is pramipexole of a pharmaceutically acceptable salt thereof.

8. The formulation of claim 7, wherein at least about 20% of the total pramipexole or pharmaceutically acceptable salt thereof is released from the formulation within the first hour as measured in an in vitro dissolution test.

9. The formulation of claim 8, wherein about 40% of the total pramipexole is released from the composition within the first two hours as measured in an in vitro dissolution test.

10. A composition for the controlled release of a dopamine agonist comprising:
the dopamine agonist or a pharmaceutically acceptable salt thereof, and a pharmaceutically acceptable excipient, said composition providing an in vitro release profile wherein at least about 20% of the dopamine agonist is released within the first hour as measured in an in vitro dissolution test, and wherein said composition provides an in vivo absorption profile wherein the time to reach 40% absorption is less than about 3 hours on average after a single dose oral administration to a human adult.

11. The composition of claim 10, wherein the dopamine agonist is pramipexole or a pharmaceutically acceptable salt thereof.

12. A method of treating a condition in a patient, comprising administering to the patient the formulation of claim 1.

13. The method of claim 12, wherein the condition is selected from Parkinson's disease and restless leg syndrome.
